(19) United States
(12) Patent Application Publication
(10) Pub. No.: US 2016/0022905 A1
(43) Pub. Date: Jan. 28, 2016

(54) DEVICE, SYSTEM AND METHOD FOR SUBCUTANEOUS DRUG DELIVERY

(71) Applicant: INSULINE MEDICAL LTD., Petah Tikva (IL)

(72) Inventors: Ron NAGAR, Tel Aviv (IL); Gabriel BITTON, Jerusalem (IL)

(73) Assignee: Insuline Medical Ltd, Petach Tikva, IL (US)

(21) Appl. No.: 14/773,298

(22) PCT Filed: Mar. 4, 2014
(86) PCT No.: PCT/IL2014/050213

§ 371 Date: Sep. 4, 2015

Related U.S. Application Data
(60) Provisional application No. 61/772,552, filed on Mar. 5, 2013.

(51) Int. Cl. A61M 5/172; A61M 5/44; A61M 37/00

(52) U.S. Cl. CPC A61M 5/1723; A61M 37/0092; A61M 5/44; A61M 2037/0007; A61M 2205/051; A61M 2205/054; A61M 2205/057; A61M 2230/005; A61M 2230/20

(57) ABSTRACT

Embodiments of the present disclosure present systems, methods and devices relate to delivering a drug subcutaneously to a patient. For example, a subcutaneous drug delivery device for use in delivering a drug subcutaneously to a patient can include a needle or cannula configured for delivery of a drug from a drug reservoir to a subcutaneous tissue of a patient.

### CROSS-REFERENCE TO RELATED APPLICATIONS

This application claims priority to U.S. Provisional Patent Application No. 61/772,552, filed Mar. 5, 2013, and entitled “Device, System and Method for Drug Delivery” the disclosure of which is incorporated herein by reference in its entirety.

### TECHNICAL FIELD

Some embodiments of the present disclosure generally relate to the administration of drugs, and in particular, to the administration of drugs subcutaneously, using a drug delivery system.

### BACKGROUND

The skin tissue is generally structured of three layers. The epidermis is the outer layer of the skin. The thickness of the epidermis varies in different types of skin. It is the thinnest on the eyelids at about 0.05 millimeters and the thickest on the palms and soles at about 1.5 millimeters. Underlying the epidermis is the dermis tissue layer. Blood vessels and nerves course through the dermis tissue layer. The dermis tissue layer also varies in thickness depending on the location of the skin. It can be about 0.3 millimeters on the eyelid and about 3 millimeters on the back.

The lowermost layer is the hypodermis tissue layer, also referred to as the subcutaneous tissue layer. The subcutaneous tissue layer is a layer of fat and connective tissue that houses larger blood vessels and nerves. The size of this layer varies throughout the body and from person to person.

Drugs can be introduced into the body of a patient by several routes of administration. There are types of drugs that are currently administrated through intravenous (IV) therapy (also referred to as “IV drip”) wherein the drug is infused directly into a vein. These intravenously administrated drugs are delivered by employing a relatively long needle with a relatively large diameter for insertion into a vein at a drug delivery site. In some IV therapies the intravenously administrated drug is injected into the vein by first accessing the vein, via the IV needle. Thereafter, a catheter connected to an IV bag, which is supported by a pole, is inserted into the vein and the IV needle is removed. The intravenously administrated drug flows from the IV bag into the vein, via the catheter.

IV therapy administration requires expertise. Frequently, a patient requires treatment by a specialized care giver at a treatment facility for a prolonged duration, until the intravenous (IV) drip treatment is complete. The patient at times is immobile during the IV therapy administration due to the cumbrous IV bag and supporting pole. Location of a vein for IV therapy administration can be difficult, especially for certain patient types such as obese, palliative-care, and neonate patients. Direct IV access also carries a risk of systemic infection. Because of the rapid effects achieved by administrating the drug directly intravenously, patients are typically observed for undesired side effects following injection.

There are types of drugs that are currently administrated by intradermal (ID) injections or infusions, such as vaccines. The intradermal administration of these intradermaly administrated drugs requires expertise since the intradermal tissue layer is superficial and is limited in volume. Consequentially, an inexperienced care giver might inadvertently inject the drug subcutaneously instead of intradermaly.

In both intravenous and intradermal drug administration, the patient is susceptible to experiencing pain and exposed to infection.

Subcutaneous (SC) drug administration is another method for drug injection or infusion. During subcutaneous administration, a needle is inserted through the epidermal and dermal tissue layers of the skin and into the fatty subcutaneous tissue. Following injection, the drug reaches the bloodstream in the circulatory system, via the capillaries and/or the lymphatic system.

In contrast to the skilled personnel required for the administration of IV and ID injections and infusions, subcutaneous drug delivery can be administered by the patient. The injections are generally unpainful and carry a reduced risk of infection and other complications. If an infectious agent is injected subcutaneously, it is generally limited to a local infection rather than a systemic infection.

However, it takes longer for a subcutaneously administrated drug to reach the circulatory system compared to IV, where the drug is administered directly to the vein, or ID therapy, when the drug is administered to the dermis, a region rich with capillaries.

### SUMMARY OF DISCLOSURE

In some embodiments, the current subject matter relates to systems, methods and devices that can provide efficient and timely delivery of a drug to the patient.

There is provided according to an embodiment of the present disclosure, a subcutaneous drug delivery device for use in delivering a drug subcutaneously to a patient. The device can include a needle or cannula configured for delivery of a drug from a drug reservoir to a subcutaneous tissue of a patient, a treatment element configured to increase delivery of the drug into the circulatory system of the patient by application of a treatment, via a surface of the skin, based on at least one property of the drug and/or at least one property of a drug depot, wherein the drug depot includes an area of tissue surrounding the needle or cannula, at least one sensor configured to generate at least one signal determinative of the at least one property and generate a sensor signal representative thereof, and a controller to receive the sensor signal and configure treatment by the treatment element based on the determined property.

In some embodiments, the application of treatment can include at least one of the following: heating, cooling, mechanical vibrations, suction, massaging, acoustic stimulation, electromagnetic radiation, magnetic stimulation, radio frequency irradiation, microwave irradiation, electrical stimulation, Transcutaneous Electrical Nerve Stimulation (TENS), an additional substance, drugs, medicament, chemicals, biologically active bacteria, biologically inactive bacteria or a combination thereof.

In some embodiments, the determined property of the drug can include a temperature of the drug in the reservoir or in the drug depot of the skin. In some embodiments, the determined property of the drug can include a concentration of the drug in the circulatory system of the patient. In some embodiments, the determined property of the drug includes a flow rate of the drug in the circulatory system of the patient.

In some embodiments, the determined property of the drug includes at least one of: an amount of drug remaining at the drug depot, a concentration of the drug in the circulatory system based upon measurement of the drug concentration remaining at the drug depot as a function of time, and a flow rate of the drug in the circulatory system based upon the measurement of the drug concentration remaining at the drug depot as a function of time.

In some embodiments, the sensor signal generates a signal of the determined property in real-time, a selected time, and/or a predetermined time. In some embodiments, the determined property of the drug includes at least one of a pharmacokinetic and pharmacodynamics profile of the drug. In some embodiments, the sensor can be provided on or adjacent the needle or cannula. In some embodiments, the sensor can be provided on or adjacent the treatment element. In some embodiments, the sensor can be spaced away from the needle or cannula. In some embodiments, the sensor can be spaced away from the treatment element. In some embodiments, the sensor includes an optical sensor or a laser Doppler flowmeter (LDF).

In some embodiments, the drug includes an intravenously and/or intradermaly administrated drug. In some embodiments, the intravenously and/or intradermaly administrated drug includes at least one of a large molecule drug, a biological drug, a cancer chemotherapy drug, a low solubility drug or a low permeability drug. In some embodiments, the needle or cannula include a gauge size greater than 24 Ga. In some embodiments, the drug can be delivered by infusion and the system further includes an infusion pump.

In some embodiments, the treatment element is configured for applying treatment to maintain a predetermined pharmacokinetic profile during the infusion of the drug. In some embodiments, the treatment element applies the treatment for maintaining a predetermined pharmacokinetic profile from the infusion of the drug up to any one of: one or more hours, one or more days, one or more weeks, and one or more months.

There is provided according to an embodiment of the present disclosure, a subcutaneous drug delivery device for use in delivering a drug subcutaneously to a patient, the device including: a drug reservoir configured to contain a drug, a needle or cannula configured for delivery of the drug from the drug reservoir to a subcutaneous tissue of a patient, a treatment element configured to increase delivery of the drug into the circulatory system of the patient by application of a treatment via the surface of the skin based on at least one property of the drug and/or a drug depot, wherein the drug depot includes an area of tissue surrounding the needle or cannula, at least one sensor configured to generate at least one signal determinative of the at least one property, and a controller to receive the sensor signal and configure treatment by the treatment element based on the determined property.

In some embodiments, the at least one property includes a blood perfusion of the drug depot and/or in proximity to the drug depot. In some embodiments, the determined property of the drug includes at least one of: an amount of drug remaining at the drug depot, a concentration of the drug in the circulatory system based upon measurement of the drug concentration remaining at the drug depot as a function of time, or a flow rate of the drug in the circulatory system based upon the measurement of the drug concentration remaining at the drug depot as a function of time.

In some embodiments, the determined blood perfusion corresponds to a degree of vasodilatation which can be induced by treatment applied by the treatment element. In some embodiments, the device can further include a second sensor configured to generate at least one signal for identifying at least one of an injection or infusion of the drug through the needle or cannula. The second sensor can be configured to identify whether the injection or infusion can be a basal or bolus dose of the drug. The controller can be further configured to at least one of apply, adjust and cease application of treatment by the treatment element depending upon the degree of vasodilatation or upon the concentration of the drug being different than a desired concentration.

In some embodiments, at least one of a pharmacokinetic and pharmacodynamic profile of the drug can be modified during delivery. The at least one of the pharmacokinetic and pharmacodynamic profiles of the drug can be modified in real-time, a selected time, and/or a predetermined time.

In some embodiments, the drug can be delivered by infusion and the system further includes an infusion pump. The treatment element may be configured to apply the treatment for maintaining a predetermined pharmacokinetic profile during the infusion of the drug. In some embodiments, the treatment element may be configured to apply the treatment for maintaining a predetermined pharmacokinetic profile from the infusion of the drug up to any one of a few hours, a day, two days, three days, a week, two weeks, a month, and a few months.

There is provided according to an embodiment of the present disclosure, a method for delivering a drug subcutaneously to a patient including providing a subcutaneous drug delivery device configured for use in delivering a drug subcutaneously to a patient, the device including a drug reservoir configured to contain a drug, a needle or cannula configured for delivery of the drug from the drug reservoir to a subcutaneous tissue of the patient, a treatment element configured to increase delivery of the drug into the circulatory system of the patient by application of a treatment via the surface of the skin based on at least one property of the drug and/or at least one property of a drug depot, wherein the drug depot includes an area of tissue surrounding the needle or cannula, at least one sensor configured to generate at least one signal determinative of the at least one property, and a controller to receive the sensor signal and configure treatment by the treatment element based on the determined property, delivering a drug subcutaneously via at least one of the needle and cannula, determining a concentration of the drug at the drug depot, and where the concentrations corresponds to a signal generated by the sensor, activating, increasing, decreasing or de-activating the treatment element upon the concentration being different than a desired concentration.

In some embodiments, at least one of determining and activating can be accomplished in at least one of: real-time, a selected time, and a predetermined time. In some embodiments, at least one of a pharmacokinetic and pharmacodynamics profile of the drug can be maintained at a desired profile. In some embodiments, maintaining the desired profile includes maintaining the concentration of the drug in the drug depot below, above, or at a predetermined concentration. In some embodiments, a plurality of treatments can be applied via the treatment element to maintain a desired pharmacokinetic and/or pharmacodynamic profile of the drug.

In some embodiments, the delivery device further includes a cooling element. In some embodiments, the treatment element includes a heater, and wherein the method further includes at least one of alternate and intermittently heating of the drug depot by the treatment element and cooling by the cooling element to control or modify at least one of a pharmacokinetic and/or pharmacodynamic profile of the drug.

In some embodiments, the treatment element can be controlled to control the concentration of the drug in the circulatory system of the patient. The sensor can be configured to detect the concentration of the drug in the patient.

There is provided according to an embodiment of the present disclosure, a subcutaneous drug delivery system, including a drug delivery device for dispensing a drug into a subcutaneous tissue of a patient, including a drug reservoir configured for containing the drug, a needle or cannula configured for delivery of the drug therethrough from the drug reservoir to the subcutaneous tissue of the patient, the drug including an intravenously and/or intradermaly administrated drug, a treatment element, where through application of treatment, the drug is delivered from the subcutaneous tissue to a circulatory system of the patient thereby improving at least one of a pharmacokinetic and pharmacodynamic property of the drug.

In some embodiments the intravenously and/or intradermaly administrated drug includes a large molecule drug. In some embodiments, the applied treatment affects the blood perfusion at the subcutaneous tissue. In some embodiments, the applied treatment affects the permeation of the drug into capillaries of the circulatory system. In some embodiments, the drug can be delivered by injection and a syringe includes the drug reservoir. In some embodiments, the drug can be delivered by infusion and the system further includes an infusion pump. In some embodiments, the treatment element may be configured to apply the treatment for maintaining a predetermined pharmacokinetic profile during the infusion of the drug. In some embodiments, the treatment element may be configured to apply the treatment for maintaining a predetermined pharmacokinetic profile from the infusion of the drug up to any one of a few hours, a day, two days, three days, a week, two weeks, a month, a few months.

In some embodiments, the system can further include a controller which may be configured for operating the treatment element. In some embodiments, the system can further include at least one sensor configured for detecting a property of the drug, the property of the drug being related to the pharmacokinetic and/or pharmacodynamic property of the drug, a controller configured for receiving a signal determinative of at least of the property of the drug (may be a digital or analog signal, which may be or include data), the treatment element provided to increase a blood perfusion of the drug into the circulatory system by applying a treatment based on the signal received by the controller.

There is provided according to an embodiment of the present disclosure, a subcutaneous drug infusion system, including a drug delivery device for infusing a drug into a subcutaneous tissue of a patient, including a drug reservoir configured for containing the drug, an infusion pump, a catheter configured to infuse the drug therethrough from the drug reservoir to the subcutaneous tissue of the patient, the drug including an intravenously and/or intradermaly administrated drug, a treatment element in communication with the infusion pump, a controller configured for receiving a signal determinative of a detected property of the drug, the treatment element, through application of treatment, the drug may be infused from the subcutaneous tissue to a circulatory system of the patient thereby improving at least one of a pharmacokinetic and pharmacodynamic property of the drug, based on the signal received by the controller.

There is provided according to an embodiment of the present disclosure, a treatment element provided for subcutaneous drug delivery, whereby the treatment element is configured to apply treatment for delivering a drug from the subcutaneous tissue of a body of a patient to the circulatory system of the patient, thereby, for example, improving at least one of a pharmacokinetic and pharmacodynamic property of the drug, the drug including an intravenously and/or intradermaly administrated drug. The intravenously and/or intradermaly administrated drug can include at least one of: a large molecule drug, a biological drug, a cancer chemotherapy drug, a low solubility drug or a low permeability drug.

There is provided according to an embodiment of the present disclosure, a method for subcutaneous delivery of a drug, including providing a drug delivery device for dispensing a drug into a subcutaneous tissue of a patient, including a drug reservoir configured for containing the drug, a needle or cannula configured for delivery of the drug therethrough from the drug reservoir to the subcutaneous tissue of the patient, the drug including an intravenously and/or intradermaly administrated drug, applying a treatment by a treatment element whereby the drug is delivered from the subcutaneous tissue to a circulatory system of the patient, resulting in improving at least one of a pharmacokinetic and pharmacodynamic property of the drug.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The principles and operations of the systems, apparatuses and methods according to some embodiments of the present disclosure may be better understood with reference to the drawings, and the following description. The drawings are given for illustrative purposes only and are not meant to be limiting.

FIG. 1 is a schematic illustration of an exemplary subcutaneous drug delivery system, according to some embodiments of the present disclosure;

FIG. 2 is a schematic illustration of an exemplary subcutaneous drug delivery system, according to some embodiments of the present disclosure; and

FIG. 3 is a schematic illustration of an exemplary subcutaneous drug delivery system, according to some embodiments of the present disclosure.

### DETAILED DESCRIPTION OF SOME OF THE EMBODIMENTS

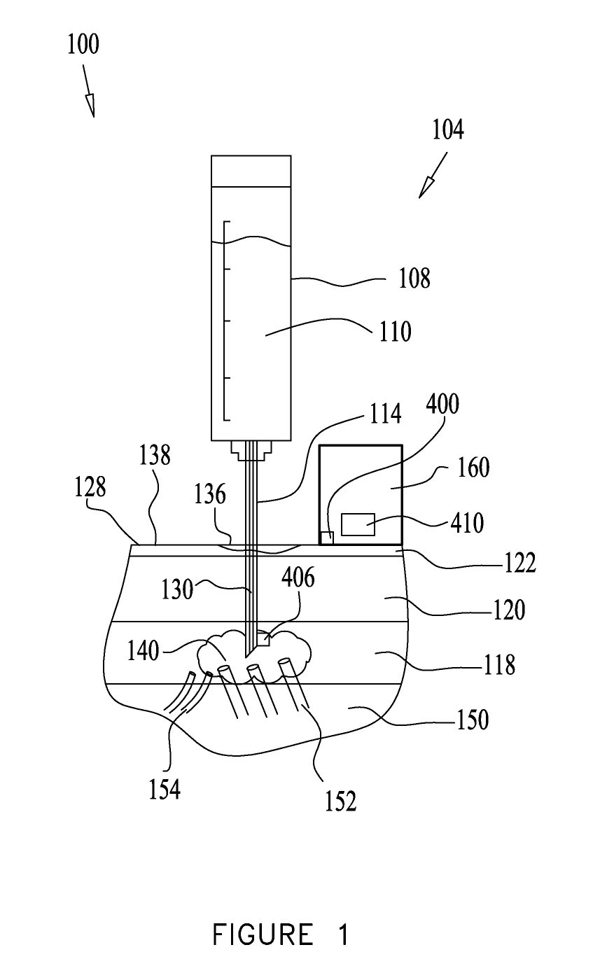

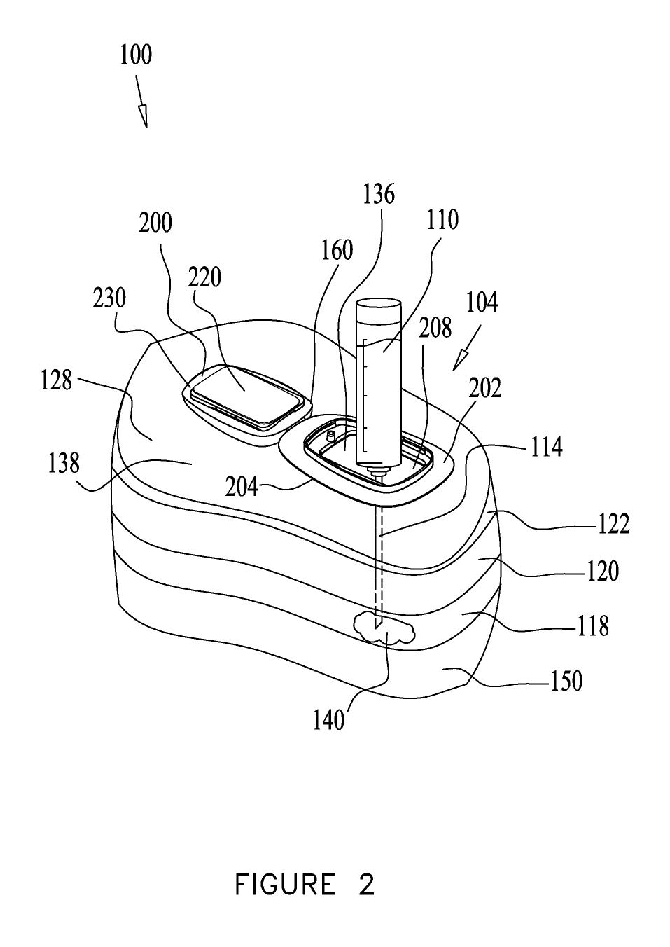

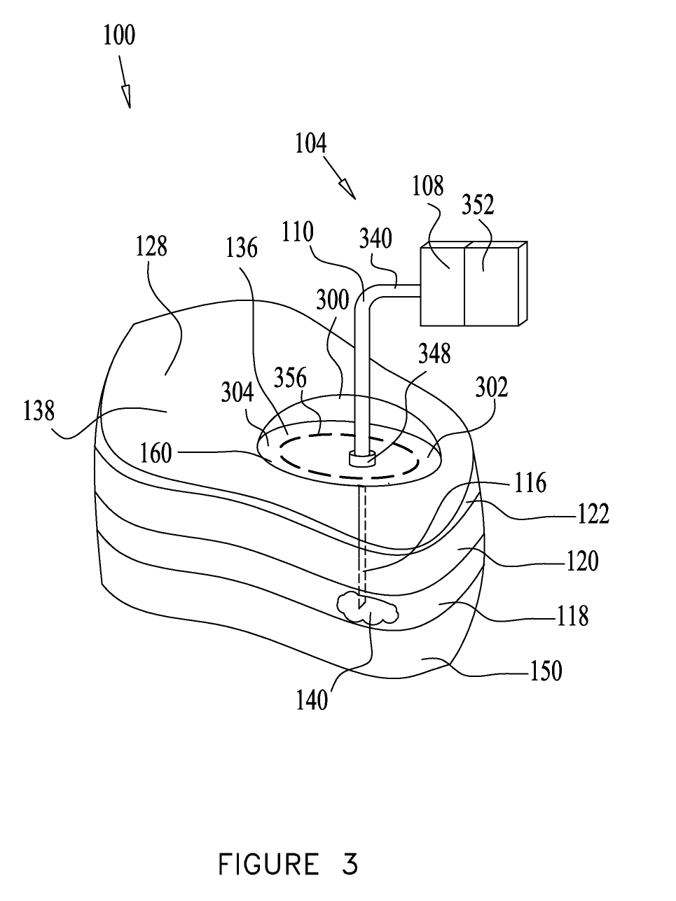

FIGS. 1-3 illustrate an exemplary subcutaneous drug delivery system 100, according to some embodiments of the present disclosure. The subcutaneous drug delivery system 100 can include a drug delivery device 104 comprising a drug reservoir 108 for containing a drug 110. A needle 114 in FIGS. 1 and 2 or a cannula 116 in FIG. 3, can deliver the drug 110 therethrough from the drug reservoir 108 to a subcutaneous tissue layer 118 of a patient.

According to some embodiments, in subcutaneous drug administration the needle 114 (FIGS. 1 and 2) can be structured in any suitable manner to reach the subcutaneous tissue layer 118, underlying a dermis tissue layer 120 and an epidermis tissue layer 122 of patient skin tissue 128.

In some embodiments, the needle 114 employed for subcutaneous drug administration is relatively short, so as to prevent injection in muscular tissues underlying the subcutaneous tissue layer 118, yet long enough to pass the dermis tissue layer 120. In a non-limiting example, the needle 114 is structured with a length in the range of about 4 millimeters to 16 millimeters. In some embodiments, the needle 114 is structured with a length of about 4 millimeters to 12 millimeters. In some embodiments, the needle 114 is structured with a length of about 8 millimeters to 12 millimeters.

In some embodiments, the needle 114 employed for subcutaneous drug administration can be relatively thin. In some embodiments, the needle gauge may be greater than 24 Ga (e.g. with a nominal inner diameter forming a duct 130 therein of about 0.311 millimeters and an outer diameter of about 0.5652 millimeters). In some embodiments, the needle gauge may be in a range between about 24-27 Ga. In some embodiments, the needle gauge may be in a range between about 25-27 Ga. In some embodiments, the needle gauge may be in a range between about 24-31 Ga.

According to an embodiment, the drug 110 is administrated by the needle 114 piercing the skin tissue 128 at a drug delivery site 136 on an outer surface 138 of the skin tissue 128. In some embodiments the drug 110 may be administrated by injection where the drug 110 flows from the reservoir 108 through the duct 130 of the needle 114 into the subcutaneous tissue layer 118.

In other embodiments, the drug 110 may be administrated by infusion where the cannula 116 (FIG. 3) can be inserted at the drug delivery site 136. The drug 110 may be infused to the subcutaneous tissue layer 118 via a catheter 340.

In both injection and infusion administration, the drug 110 reaches a drug depot 140. The drug 110 flows thereafter into the circulatory system 150, via capillaries 152 and/or the lymphatic system 154.

In some embodiments, the drug depot 140 may comprise and area of tissue surrounding the needle 114 or cannula 116. This tissue may comprise subcutaneous tissue 118.

It is noted that the skin tissue 128, layers thereof and drug delivery device 104 may not be shown to scale in FIGS. 1-3.

According to an embodiment of the disclosure, the subcutaneous drug delivery system 100 may comprise a treatment element 160. The treatment element 160, through application of treatment, can improve the pharmacokinetic and/or pharmacodynamic property of the drug 110 for delivering the drug 110 from the subcutaneous tissue layer 118 to the circulatory system 150.

In some embodiments, the treatment element 160 can be configured to apply any suitable treatment capable of enhancing a tissue response to the delivered drug 110.

In some embodiments, the treatment element 160 can be configured to increase delivery of the drug 110 into the circulatory system 150 by application of a treatment, via the surface of the skin 138, based on at least one property of the drug 110 and/or at least one property of the drug depot 140.

The treatment element 160 can be placed at any suitable location. For example, the treatment element 160 can be placed on the skin surface 138 or in proximity thereto. In some embodiments, the treatment element 160 can be placed in proximity to the drug delivery site 136. In some embodiments, the treatment element 160 can be placed away from the drug delivery site 136.

The treatment applied by the treatment element 160 can include, but not be limited to, for example, any one of: electrical, magnetic and/or mechanical stimulus, such as heating, cooling, mechanical vibrations, suction, massaging, acoustic stimulation (e.g., ultrasound), electromagnetic radiation, electric field stimulation, magnetic field stimulation, radio frequency irradiation, microwave irradiation, electrical stimulation, magnetic stimulation, Transcutaneous Electrical Nerve Stimulation (“TENS”), or the like, and/or any combination of the above treatments to improve the drug's pharmacokinetic profile and/or pharmacodynamic profile. In some embodiments, the treatment element 160 can stimulate or inhibit tissue by introducing additional substances (in addition to the therapeutic fluid), for example, including, but not limited to, drugs, medicament, chemicals, biologically active bacteria, biologically inactive bacteria or the like or also any combination of the above treatments to improve the drug's pharmacokinetic profile and/or pharmacodynamic profile.

In some embodiments, the application of treatment by the treatment element 160 can affect a degree of blood perfusion in the vicinity of the drug delivery site 136 and/or the drug depot 140, which in turn, can affect the delivery of the drug 110 to the drug depot 140 and/or into the circulatory system 150. Accordingly, increasing the blood perfusion by the treatment element 160 can increase the amount of drug 110 delivered to the drug depot 140 and/or into the circulatory system 150. Accordingly, the treatment element 160 may be configured to increase delivery of the drug 110 into the circulatory system 150 by application of a treatment via the skin surface 138 based on a property of the drug 110.

In some embodiments, the application of treatment by the treatment element 160 can affect a degree of vasodilation, which in turn, can affect the permeability of the drug 110 into the capillaries 152 and thus the further delivery of the drug 110 to a desired target site within the body.

In some embodiments, the application of treatment by the treatment element 160 can affect a degree of vasodilation, which in turn, can affect the permeability of interstitial fluids (ISF) into the capillaries 152. Increase of interstitial fluid permeability can increase the fluidity at the drug depot 140, which can increase a rate of the drug delivery from the drug depot 140 into the circulatory system 150.

In some embodiments, the applied treatment can reduce variability of the drug absorption in the circulatory system 150, the blood, the capillaries 152, and/or lymph system 154 and/or its local and/or systemic effects. For example, heating the tissue in the drug delivery site 136 to a preset regulated temperature before, during and/or after the drug infusion or injection and absorption into the blood, can make blood perfusion at the drug depot 140 more reproducible and the drug absorption process more uniform and reproducible as well. Also, by reducing the delay between the drug delivery into the skin tissue and absorption into the circulatory system 150, the variability of the drug action induced by the delayed peak action profile can be reduced.

The treatment element 160 can be applied in any suitable manner. Exemplary embodiments are shown in FIGS. 2 and 3, though it is appreciated that the treatment element 160 may be applied in any suitable manner and configuration.

As seen in FIG. 2, the drug 110 is shown delivered by injection. The treatment element 160 may comprise a device 200 comprising a first unit 202, which can comprise a lower surface 204 having a biocompatible adhesive for coupling the first unit 202 to the skin surface 138. The first unit 202 can be formed with an aperture 208 overlying the skin surface 138 at the drug delivery site 136 and for allowing the needle 114 to be inserted therethrough into the subcutaneous tissue layer 118. In the embodiment shown in FIG. 2, the treatment can be applied in a form of heat provided by a heating element 220. The heating element 220 may be applied to the skin surface 138 before, during and/or after the injection of the drug 110 is administrated. The device 200 may remain on the skin surface 138 for a selected time period. Further injections of the drug 110 may be administrated at the drug delivery site 136 through aperture 208.

The heating element 220 can be placed in any suitable location. In some embodiments, the heating element 220 can be embedded in a second unit 230, coupled to the first unit 202, as seen in FIG. 2. In some embodiments, the heating element 220 can be placed in the first unit 202 and then the second unit 230 can be obviated.

In some embodiments, the drug delivery device 104 can be configured as a syringe, as shown in FIG. 1. In some embodiments, the drug delivery device 104 can be configured as an injection pen.

Another exemplary treatment element 160 is shown in FIG. 3 where the drug 110 is shown delivered by infusion. A device 300 can comprise the treatment element 160. The device 300 can comprise a lower surface 304 having a biocompatible adhesive for coupling the device 300 to the skin surface 138. The device 300 can be configured to be placed on the skin surface 138 at the drug delivery site 136. The device 300 can comprise the catheter 340 formed, on one end thereof, with the cannula 116, which can be inserted into the subcutaneous tissue layer 118. In some embodiments, a connector 348 can connect the catheter 340 to the skin tissue 128.

In some embodiments the catheter 340 can be connected at a second end thereof to the drug reservoir 108. In some embodiments, the device 300 can comprise an infusion pump 352, provided for control of the drug delivery from the drug reservoir 108. In other embodiments the infusion pump 352 may be obviated.

The treatment element 160 can be placed in any suitable location. As seen in FIG. 3, the treatment element 160 can be configured in the device 300 and can be connected to the catheter 340. In some embodiments, the treatment element 160 can be disconnected from the catheter 340. In some embodiments, the treatment element 160 can be placed on the catheter 340 or in proximity thereto.

In a non-limiting example, such as shown in FIG. 3, the treatment can be applied in a form of heat provided by a heating element 356 within the device 300. The heating element 358 may be applied to the skin surface 138 before, during and/or after the infusion of the drug 110 is administrated. The device 300 may remain on the skin surface 138 for a selected time period. In some embodiments, this selected time period can be prior to the infusion, during the infusion or a portion thereof and/or after the infusion is completed.

In some embodiments, the catheter 340 can be connected to a bag containing the drug 110.

In some embodiments, the treatment element 160 can include a treatment device disclosed in any one of co-owned International Patent Application Nos. PCT/IB2008/051044; PCT/IB2008/051046; PCT/IB2008/051049; PCT/IB2008/051050; PCT/IB2008/003547; PCT/IB2009/007600; PCT/IB2010/054476; PCT/IL2010/000623; PCT/IB2012/052335; PCT/IL2012/000211 the disclosures of which are incorporated herein by reference in their entireties.

In some embodiments, the subcutaneous drug delivery system 100 can comprise a sensor 400, such as shown in FIG. 1. The sensor 400 can be configured for detecting a property of the drug 110 at the drug depot 140 and for providing a signal indicative or determinative of the property. For example, the detected or determined property can be the drug 110 temperature, the drug 110 concentration in the circulatory system 150, the drug 110 flow rate in the circulatory system 150, the amount of drug 110 remaining at the drug depot 140 at any selected time, or any indication of the pharmacokinetic and/or pharmacodynamics profile of the drug 110. Additionally, for example, the detected or determined property of the drug 110 can comprise the concentration or a flow rate of the drug in the circulatory system 150 of the patient based upon measurement of the drug concentration remaining at the drug depot 140 as a function of time, which may include determining the concentration or flow rate of the drug 110 along a desired time period or duration.

In some embodiments, sensor 400 may be configured to generate at least one signal determinative of at least one property of the drug 110 and generate a sensor signal representative thereof.

The sensor can 400 be placed at any suitable location, such as on the skin surface 138 and/or on any suitable location in proximity to the drug depot 140. In some embodiment, the sensor 400 can be embedded in the treatment element 160. In some embodiment, the sensor 400 can be placed on or adjacent the treatment element 160 or spaced away from the treatment element 160. In some embodiments, the sensor 400 may be placed on or adjacent the needle 114 or cannula 116 or spaced away from the needle 114 or cannula 116.

In some embodiment, the sensor 400 can be configured to detect a temperature of the skin surface 138 and accordingly determine the temperature of the drug 110 at the drug depot 140 and/or in the circulatory system 150.

In some embodiment, the sensor 400 can be configured to measure local blood perfusion in proximity to the drug depot 140 and accordingly identify a degree of vasodilatation, which is induced by treatment applied by the treatment element 160. For example, the sensor 400 can comprise an optical sensor that measures optical properties of the tissue surface 138, or a Laser Doppler Flowmeter (“LDF”) that can measure local blood perfusion in proximity to the drug depot 140.

In some embodiment, the sensor 400 can be configured to generate a signal determinative of a property of a drug depot 140. In some embodiments, the property may comprise a blood perfusion of the drug depot 140 and/or in proximity to the drug depot 140. The determined blood perfusion may correspond to a degree of vasodilatation which is induced by treatment applied by the treatment element 160.

In some embodiments, the sensor 400 can comprise an injection or infusion detection sensor. In some embodiments, the sensor 400 can detect the amount of other properties including information related to the drug 110, such as the dose, duration, frequency, flow rate and/or temperature of the drug 110. In some embodiments, the sensor 400 can comprise an injection or infusion detection sensor, which can be configured as a bolus or basal dose detection element.

In some embodiments, the sensor 400 can generate a signal of the determined property of the drug 110 in real-time, at a selected time, and/or a predetermined time.

In some embodiments, a second sensor 406 can be provided. In some embodiments, the second sensor 406 may be configured to generate a signal for identifying an injection or infusion of the drug 110 through the needle 114 or cannula 116. In some embodiments, second sensor 406 may be configured to identify whether the injection or infusion is a basal or bolus dose of the drug 110.

The second sensor 406 can be placed at any suitable location, such as on the skin surface 138 and/or on any suitable location in proximity to the drug depot 140. In some embodiment, the second sensor 406 can be embedded in the treatment element 160. In some embodiment, the second sensor 406 can be placed on or adjacent the treatment element 160 or spaced away from the treatment element 160. In some embodiments, the second sensor 406 can be placed on or adjacent the needle 114 or cannula 116 or spaced away from the needle 114 or cannula 116.

In some embodiments, the subcutaneous drug delivery system 100 can comprise a controller 410.

In some embodiments, the controller 410 can be configured to receive the signal from the sensor 400 and/or second sensor 406. Based in the received signal, the controller 410 can be configured to apply the treatment by the treatment element 160.

In some embodiments, the controller 410 can receive the sensor signal and can configure treatment by the treatment element 160 based on the detected property.

In a non-limiting example, when the treatment element 160 applies heat, the sensor 400 can detect the degree of vasodilatation induced by the heat in proximity to the drug depot 140. Accordingly, the degree of vasodilation can indicate the degree of the drug profusion into the circulatory system 150. Based on the degree of vasodilatation, the controller 410 may apply further heat to induce further profusion of drug 110 into the circulatory system 150. Alternatively, based on the degree of vasodilatation, the controller 410 may cease applying further heat to cease, or impede further profusion of drug 110 into the circulatory system 150.

In some embodiments, the controller 410 can be configured to apply the treatment, in response to other components besides the sensor 400, such as in response to the operation of the pump 352 of FIG. 3, for example.

According to an embodiment, the drug 110 comprises a type of drug that is typically intravenously administrated and/or a type of drug that is typically intradermaly administrated.

In some embodiments, intravenously administrated drugs require relatively large dose delivery, relatively precise dosage delivery or rapid delivery and thus are administrated directly into the vein.

In some embodiments, relatively large dose delivery may comprise doses larger than about 1 or 2 milliliters, for example.

A non-limiting example of drugs typically requiring relatively precise dosage delivery or rapid delivery can include biologics, cancer chemotherapy drugs, drugs comprising irritating agents such as cytotoxics, for example.

Non-limiting examples of cancer chemotherapy drugs may include any one of the following: carboplatin, cisplatin, cyclophosphamide, docetaxel, doxorubicin, etoposide, fluorouracil, gemcitabine, irinotecan, methotrexate, paclitaxel, sunitinib, topotecan, vincristine and vinblastine.

The drug delivery system 100 is configured to deliver the intravenously administrated drugs by subcutaneous administration methods, while maintaining the requirements of administrating relatively large doses, relatively precise dosage delivery or rapid delivery. In some embodiments, the treatment element 160 is configured to modify the pharmacokinetic profile and/or pharmacodynamic profile of the intravenously administrated drug 110, thereby enabling the relatively large dose delivery, relatively precise dosage delivery and/or rapid delivery of the drug 110.

In a non-limiting example, the pharmacokinetic profile and/or pharmacodynamic profile of the drug 110 may be modified by application of an electrical or mechanical stimulus. The electrical or mechanical stimulus can increase the blood perfusion in the vicinity of the drug delivery site 136, which increases the flow of the drug 110 to the drug depot 140 and into the circulatory system 150. Additionally, the electrical or mechanical stimulus can increase the vasodilation of the capillaries 152 which allows the drug 110 to rapidly enter the capillaries 152. This increase of blood perfusion and vasodilation provides for rapid delivery and accordingly large dose delivery. The ability to control the degree of blood perfusion and vasodilation by the electrical or mechanical stimulus allows for relatively precise dosage delivery.

In some embodiments of the present disclosure, the pharmacokinetic and/or pharmacodynamics profile of the drug 110 can be controlled to a greater degree during the subcutaneous administration by the drug delivery system 100 than during intravenous administration. In some embodiments of the present disclosure, the pharmacokinetic and/or pharmacodynamics profile of the drug 110 can be controlled and appropriately modified during the subcutaneous drug delivery (namely at “real-time”) by the drug delivery system 100. Real-time modification is conventionally infeasible during conventional intravenous delivery.

In some embodiments, the delivery of the drug refers to the duration from the introduction of the drug 110 into the body and until the drug is transferred into the circulatory system 150 and on to a desired target site within the body.

In a non-limiting example, during the subcutaneous drug delivery, the sensor 400 (or sensor 406) can detect the concentration of the drug 110 at the drug depot 140. A signal determinative of the drug concentration can be transmitted to the controller 410. Wherein the concentration of the drug 110 is found to be lower than a desired concentration, the controller may activate the treatment element to apply heat, thereby increasing the drug delivery into the circulatory system 150. During the drug delivery, the sensor 400 (or sensor 406) can continually detect the properties of the drug 110 and accordingly the controller 410 can adjust the applied treatment, such as by heating or cooling, to deliver the drug at the desired concentration.

Real-time adjustment of the properties of the drug 110 during subcutaneous delivery, can be advantageous particularly due to the dynamic changes in the properties (e.g. temperature, blood flow rate) of the skin tissue 138 and circulatory system 150 in accordance with the body's physiological condition (e.g. physical activity, illness). Accordingly the pharmacokinetic and/or pharmacodynamics profile of the drug 110 can be controlled and maintained at a desired profile. In some embodiments, the pharmacokinetic and/or pharmacodynamic profiles of the drug 110 can be modified in real-time, at selected time, and/or at a predetermined time.

In some embodiments, the treatment element 160 may apply the treatment for maintaining a predetermined pharmacokinetic profile from the time of the infusion of the drug 110 for a long time period, such as up to any one of: a few hours; a day, two days, three days, a week; two weeks; a month, a few months. This lengthy maintenance of the predetermined profile may be provided by the pump 352, which can be configured to service the patient for a long time.

In a non-limiting example the desired or predetermined profile can include maintaining the concentration of the drug 110 above a predetermined concentration. In a non-limiting example, the desired profile can include maintaining the concentration of the drug 110 below a predetermined concentration. In a non-limiting example the desired profile can include maintaining the concentration of the drug 110 about equally to a predetermined concentration.

Accordingly, is some embodiments of the invention, the treatment element 160 can be configured to apply more than one treatment so as to maintain the desired pharmacokinetic and/or pharmacodynamics profile of the drug 110. For example, the device 200 of FIG. 2 can comprise a cooling element in additional to the heating element 220 for intermittently heating and cooling to the skin surface 138.

Real-time adjustment of the properties of the drug 110 during subcutaneous delivery (which may also be referred to as “profile on demand”), can be advantageous in drugs 110 comprising intravenously administrated drugs, containing toxins or harmful drugs, when administrated above a predetermined amount or concentration. An example for drugs 110 containing toxins can be cancer chemotherapy drugs. In these toxic drugs it is of paramount importance to maintain the predetermined pharmacokinetic and/or pharmacodynamics profile and prevent the drug concentration in the body from exceeding the predetermined amount. Additionally, since subcutaneous drug delivery is less painful than intravenous drug delivery and can also be easily administrated at different locations in the body, it is greatly advantageous to subcutaneously deliver toxin containing drugs by employing the drug delivery system 100.

Moreover, real-time adjustment of the properties of the drug 110 during subcutaneous delivery, can be advantageous in intravenously administrated drugs 110, which are infused, such as shown in FIG. 3. During infusion, the treatment element 160 can apply the treatment to maintain a desired pharmacokinetic and/or pharmacodynamics profile of the infused drug 110. For example, the treatment element 160 can apply the treatment substantially during all the duration of the infusion, to maintain a constant concentration of the infused, drugs 110 in the circulatory system 150.

In some embodiments, the sensor 400 can be configured to detect the concentration of the infused drugs 110 and accordingly the controller 410 can apply the treatment. In other embodiments, the controller 410 can be configured to apply the treatment for maintaining the constant concentration, in response to other components besides the sensor 400, such as in response to the operation of the pump 352, for example.

In some embodiments, the pharmacokinetic profile comprises the concentration of the drug 110 during delivery thereof into the body. The pharmacodynamics profile can comprise the effect (e.g. biochemical and physiological) the drug 110 has the on body during delivery thereof into the body.

In some embodiments, these types of intravenously administrated drugs can comprise large molecule drugs. In some embodiments, large molecule drugs can be based on molecules larger than 900 Daltons.

In some embodiments, large molecule drugs can comprise biopharmaceutical drugs based on biological components. In some embodiments the biopharmaceutical drugs are based on proteins that have a therapeutic effect. These large protein molecules, which can be composed of more than 1,300 amino acids and can be as heavy as 150,000 g/mol (or 150 kDa), for example, can be essentially copies or optimized versions of endogenous human proteins. Biologics bind to specific cell receptors that are associated with the disease process. Monoclonal antibodies are specialized in recognizing a very specific structure on the cell surface. For example, these biopharmaceutical drugs can be used in cancer therapy. These biopharmaceutical drugs can bind selectively, for example, to the receptors of cancer cells, making it possible to mark and fight specific abnormal cells. Healthy cells are usually not attacked in this process, so that biologics often cause fewer side effects than in conventional chemotherapy.

Due to the relatively large size of the large molecule drugs, these large molecule drugs are conventionally administrated intravenously due to a relatively small lumen of the capillaries 152 or other tissues or organs, which can prevent the large molecule drugs from passing therethrough. According to some embodiments, the drug delivery system 100 is configured to deliver the large molecule drugs by subcutaneous administration methods, while allowing the large molecule drugs to pass through the capillaries 152 or other tissues or organs.

In a non-limiting example, the treatment element 160 can apply an electrical or mechanical stimulus. The electrical or mechanical stimulus can increase the blood perfusion in the vicinity of the drug delivery site 136 which increases the flow of the large molecule drugs to the drug depot 140 and into the circulatory system 150. Additionally, the electrical or mechanical stimulus can increase the vasodilation of the capillaries 152 or other tissues or organs, which allows the large molecule drugs to enter the capillaries 152 or other tissues or organs. This increase of blood perfusion and vasodilation provides for delivery of the large molecule drugs.

In some embodiments, the drugs 110 can include relatively low solubility drugs and/or low permeability drugs. These low solubility drugs and/or low permeability drugs, when trapped at the drug depot 140 can be degraded, leading to lowered bioavailability.

The drug delivery system 100 is configured to deliver the low solubility drugs and/or low permeability drugs by subcutaneous administration methods, while preventing the trapping of these drugs at the drug depot 140. In some embodiments, the treatment element 160 is configured to modify the pharmacokinetic profile and/or pharmacodynamic profile of the low solubility drugs and/or low permeability drugs, thereby enabling their delivery into the circulatory system 150.

In a non-limiting example, the pharmacokinetic profile and/or pharmacodynamic profile of the low solubility drugs and/or low permeability drugs may be modified by application of an electrical or mechanical stimulus. The electrical or mechanical stimulus can increase the blood perfusion in the vicinity of the drug delivery site 136, which increases the flow of the low solubility drugs and/or low permeability drugs to the drug depot 140 and into the circulatory system 150. Additionally, the electrical or mechanical stimulus can increase the vasodilation of the capillaries 152 which allows the low solubility drugs and/or low permeability drugs to rapidly enter the capillaries 152. This increase of blood perfusion and vasodilation provides for efficient delivery of the low solubility drugs and/or low permeability drugs.

In some embodiments, the drug delivery system 100 is configured to deliver intradermaly administrated drugs by subcutaneous administration methods, while maintaining the requirements of administrating relatively large dose delivery, relatively precise dosage delivery or rapid delivery. In some embodiments, the treatment element 160 is configured to modify the pharmacokinetic profile and/or pharmacodynamic profile of the drug 110, thereby enabling relatively large dose delivery, relatively precise dosage delivery and/or rapid delivery of the drug 110.

In a non-limiting example, the pharmacokinetic profile and/or pharmacodynamic profile of the drug 110 may be modified by application of an electrical or mechanical stimulus. The electrical or mechanical stimulus can increase the blood perfusion in the vicinity of the drug delivery site 136 which increases the flow of the drug 110 to the drug depot 140 and into the circulatory system 150. Additionally, the electrical or mechanical stimulus can increase the vasodilation of the capillaries 152 which allows the drug 110 to rapidly enter the capillaries 152. This increase of blood perfusion and vasodilation provides for rapid delivery and accordingly large dose delivery. The ability to control the degree of blood perfusion and vasodilation by the electrical or mechanical stimulus allows for relatively precise dosage delivery.

In some embodiments of the present disclosure, the pharmacokinetic and/or pharmacodynamics profile of the drug 110 can be controlled to a greater degree during the subcutaneous administration by the drug delivery system 100 than during intradermal administration. In some embodiments of the present disclosure, the pharmacokinetic and/or pharmacodynamics profile of the drug 110 can be controlled and appropriately modified during the subcutaneous drug delivery (namely at “real-time”) by the drug delivery system 100. Real-time modification is conventionally infeasible during conventional intradermal delivery.

Real-time adjustment of the properties of the intradermaly administrated drug 110 during subcutaneous delivery, can be advantageous particularly due to the dynamic changes in the properties (e.g. temperature, blood flow rate) of the skin tissue 138 and circulatory system 150 in accordance with the body's physiological condition (e.g. physical activity, illness). Accordingly the pharmacokinetic and/or pharmacodynamics profile of the drug 110 can be controlled and maintained at a desired profile.

In some embodiments, the treatment element 160 can be applied in combination with other methods for increased bold perfusion. Such a method can include use of a pharmaceutical or agent, such as the recombinant Human Hyaluronidase, for example.

In some embodiments, the treatment element 160 can include a non-pharmaceutical treatment. In a non-limiting example, this treatment may comprise an electrical or mechanical stimulus. There may be an advantage in applying a non-pharmaceutical treatment, since there is no requirement to ensure that the applied treatment is compatible with the drug 110 and there is no risk in the combination thereof.

In some embodiments, the drug delivery site 136 can be a tissue deeper than the subcutaneous tissue layer 118, such as within any organ or viscera and the treatment element 160 may be configured to apply additional treatment or stimulation to the vicinity of the drug delivery site 136, as described above.

In some embodiments the drug delivery system 100 can comprise further mechanical and/or electrical components and connections.

Communication between the sensor 400, the controller 410 and any other components of the treatment element 160 or a component of the drug delivery system 100 can be provided in any suitable manner. In some embodiments, the communication can be wired and provided through electrical connections. In some embodiments, the communication can be wireless via an analog short range communication mode, or a digital communication mode including WIFI or BLUETOOTH®. Additional examples of such communication can include a network. The network can include a local area network (“LAN”), a wide area network (“WAN”), or a global network, for example. The network can be part of, and/or can include any suitable networking system, such as the Internet, for example, and/or an Intranet. Generally, the term “Internet” may refer to the worldwide collection of networks, gateways, routers, and computers that use Transmission Control Protocol/Internet Protocol (“TCP/IP”) and/or other packet based protocols to communicate therebetween.

In some embodiments the drug delivery system 100 may comprise a single or plurality of transmission elements for communication between components thereof. In some embodiments, the transmission element can include at least one of the following: a wireless transponder, or a radio-frequency identification (“RFID”) device. The transmission element can include at least one of the following, for example: a transmitter, a transponder, an antenna, a transducer, and/or an RLC circuit or any suitable components for detecting, processing, storing and/or transmitting a signal, such as electrical circuitry, an analog-to-digital (“A/D”) converter, and/or an electrical circuit for analog or digital short range communication.

In some embodiments, the controller 410 and/or any other relevant component of the drug delivery system 100 can include a processor, a memory, a storage device, and an input/output device.

Various implementations of some of embodiments disclosed, in particular at least some of the processes discussed (or portions thereof), may be realized in digital electronic circuitry, integrated circuitry, specially configured ASICs (application specific integrated circuits), computer hardware, firmware, software, and/or combinations thereof. These various implementations, such as associated with the drug delivery system 100 and the components thereof, for example, may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs, software, software applications or code) include machine instructions/code for a programmable processor, for example, and may be implemented in a high-level procedural and/or object-oriented programming language, and/or in assembly/machine language. As used herein, the term “machine-readable medium” refers to any computer program product, apparatus and/or device (e.g., non-transitory mediums including, for example, magnetic discs, optical disks, flash memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a LCD (liquid crystal display) monitor and the like) for displaying information to the user and a keyboard and/or a pointing device (e.g., a mouse or a trackball, touchscreen) by which the user may provide input to the computer. For example, this program can be stored, executed and operated by the dispensing unit, remote control, PC, laptop, smartphone, media player or personal data assistant (“PDA”). Other kinds of devices may be used to provide for interaction with a user as well. For example, feedback provided to the user may be any form of sensory feedback (e.g., visual feedback, auditory feedback, or tactile feedback), and input from the user may be received in any form, including acoustic, speech, or tactile input. Certain embodiments of the subject matter described herein may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components.

The components of the system may be interconnected by any form or medium of digital data communication (e.g., a communication network). Examples of communication networks include a local area network (“LAN”), a wide area network (“WAN”), and the Internet. The computing system according to some such embodiments described above may include clients and servers. A client and server are generally remote from each other and typically interact through a communication network. The relationship of client and server arises by virtue of computer programs running on the respective computers and having a client-server relation to each other.

Any and all references to publications or other documents, including but not limited to, patents, patent applications, articles, webpages, books, etc., presented anywhere in the present application, are herein incorporated by reference in their entirety.

Example embodiments of the devices, systems and methods have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements/features from any other disclosed methods, systems, and devices, including any and all features corresponding to translocation control. In other words, features from one and/or another disclosed embodiment may be interchangeable with features from other disclosed embodiments, which, in turn, correspond to yet other embodiments. Furthermore, one or more features/elements of disclosed embodiments may be removed and still result in patentable subject matter (and thus, resulting in yet more embodiments of the subject disclosure).

1-63. (canceled)

64. A subcutaneous drug delivery device for use in delivering a drug subcutaneously to a patient, the device comprising:
a needle or cannula configured for delivery of a drug from a drug reservoir to a subcutaneous tissue of a patient;
a treatment element configured to modify delivery of the drug into the circulatory system of the patient by application of a treatment via a surface of skin based on at least one property of the drug and/or at least one property of a drug depot, wherein the drug depot comprises an area of tissue surrounding the needle or cannula;
at least one sensor configured to generate at least one signal determinative of the at least one property and generate a sensor signal representative thereof; and
a controller to receive the sensor signal and configure treatment by the treatment element based on the at least one property, wherein the at least one property of the drug comprises at least one of a pharmacokinetic and pharmacodynamics profile of the drug.

65. The device of claim 64, wherein the application of treatment includes at least one of the following: heating, cooling, mechanical vibrations, suction, massaging, acoustic stimulation, electromagnetic radiation, magnetic stimulation, radio frequency irradiation, microwave irradiation, electrical stimulation, Transcutaneous Electrical Nerve Stimulation (TENS), an additional substance, drugs, medicament, chemicals, biologically active bacteria, biologically inactive bacteria or a combination thereof.

66. The device of claim 64, wherein the determined property of the drug comprises at least one of:
an amount of drug remaining at the drug depot;
a concentration of the drug in the circulatory system based upon measurement of the drug concentration remaining at the drug depot as a function of time; and
a flow rate of the drug in the circulatory system based upon the measurement of the drug concentration remaining at the drug depot as a function of time.

67. The device of claim 64, wherein the sensor is at least one of the following:
provided on or adjacent the needle or cannula;
provided on or adjacent the treatment element;
spaced away from the needle or cannula; and
spaced away from the treatment element.

68. The device of claim 64, further comprising a second sensor configured to perform at least one of the following:
generate at least one signal for identifying at least one of an injection or infusion of the drug through the needle or cannula; and
identify whether the injection or infusion is a basal or bolus dose of the drug.

69. The device of claim 64, wherein the drug comprises an intravenously and/or intradermaly administrated drug.

70. The device of claim 69, wherein the intravenously and/or intradermaly administrated drug comprises a large molecule drug.

71. The device of claim 69, wherein the intravenously and/or intradermaly administrated drug comprises a biological drug.

72. The device of claim 69, wherein the intravenously and/or intradermaly administrated drug comprises a cancer chemotherapy drug.

73. The device of claim 69, wherein the intravenously and/or intradermaly administrated drug comprises a low solubility drug and/or a low permeability drug.

74. The device of claim 69, wherein the intravenously and/or intradermaly administrated drug comprises a drug configured for large dose delivery.

75. The device of claim 69, wherein the intravenously and/or intradermaly administrated drug comprises an irritating agent or toxic drug or drug configured for rapid delivery.

76. The device of claim 64, wherein the treatment element applies the treatment for maintaining a predetermined pharmacokinetic profile from the delivery of the drug up to any one of: a few hours, a day, two days, three days, a week, two weeks, a month, and a few months.

77. The device of claim 64, wherein the at least one property comprises a blood perfusion of the drug depot and/or in proximity to the drug depot.

78. The device of claim 77, wherein the determined blood perfusion corresponds to a degree of vasodilatation which is induced by treatment applied by the treatment element.

79. A method for delivering a drug subcutaneously to a patient comprising:
providing a subcutaneous drug delivery device configured to deliver a drug subcutaneously to a patient, the device comprising:
a drug reservoir configured to contain a drug;
a needle or cannula configured for delivery of the drug from the drug reservoir to a subcutaneous tissue of the patient;
a treatment element configured to modify delivery of the drug into the circulatory system of the patient by application of a treatment via a surface of skin based on at least one property of the drug and/or at least one property of a drug depot, wherein the drug depot comprises an area of tissue surrounding the needle or cannula;
at least one sensor configured to generate at least one signal determinative of the at least one property; and
a controller to receive the at least one signal and configure treatment by the treatment element based on the at least one property;
delivering the drug subcutaneously via at least one of the needle and cannula;
determining a concentration of the drug at the drug depot, and wherein the concentration corresponds to at least one signal generated by the sensor; and
activating, increasing, decreasing or de-activating the treatment element upon the concentration being different than a desired concentration.

80. The method of claim 79, wherein at least one of a pharmacokinetic and pharmacodynamics profile of the drug is maintained at a desired profile.

81. The method of claim 80, wherein maintaining the desired profile includes maintaining the concentration of the drug in the drug depot below, above, or at a predetermined concentration.

82. The method of claim 79, wherein treatment element comprises a heater and a cooling element, and wherein the method further comprises at least one of alternately and intermittently heating the drug depot by the heater and cooling the drug depot by the cooling element to control or modify at least one of a pharmacokinetic and/or pharmacodynamic profile of the drug.

83. The method of claim 79, wherein the treatment element is controlled to control the concentration of the drug in the circulatory system of the patient.
